(19) United States
(12) Patent Application Publication
(10) Pub. No.: US 2008/0220000 A1
(43) Pub. Date: Sep. 11, 2008

(54) Methods for Generating Improved Immune Reponse

(76) Inventors: Anne Clare Moore, Cork (IE); Adrian V.S. Hill, Oxford (GB)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 11/579,190

(22) PCT Filed: May 3, 2005
(86) PCT No.: PCT/GB05/01677

§ 371 Date: Sep. 18, 2007

(30) Foreign Application Priority Data
Apr. 30, 2004 (GB) 0409799.4

(51) Int. Cl. A61K 39/395; A61K 39/00; A61P 37/00

(52) U.S. Cl. 424/172.1; 424/278.1; 424/199.1

(57) ABSTRACT

This application relates to a method for generating an improved immune response in a host. The method involves the step of administering a vectored vaccine in the presence of an agent that impairs Treg cell function.

The present invention relates to a method for generating an improved immune response in a host. The method involves the step of administering a vectored vaccine in the presence of an agent that impairs Treg cell function.

All publications, patents and patent applications cited herein are incorporated in full by reference.

### BACKGROUND TO THE INVENTION

Immune responses must be qualitatively and quantitatively controlled within limits to prevent the induction of immune pathology. In recent years, it has been discovered that a subset of T cells, identified as CD4⁺CD25⁺ T cells and termed regulatory T cells (herein, Treg), are crucial to the regulation of the magnitude and specificity of immunity to self antigens. Dysfunction of this Treg population can result in auto-immunity, where the immune system responds to a self antigen and auto-reactive lymphocytes, free from Treg control, destroy the host's own tissues (1-5). Functional Tregs have been shown to be a barrier to cancer immunotherapies, where the aim is to force the immune system to mount a response against the tumour self-antigen. Depleting these populations of Treg has been demonstrated to improve significantly the clearance of injected tumour cells (6-9). Improving the regulatory capacity of Treg has also demonstrated some promise in inducing immune tolerance to transplanted tissues (10). These diverse studies have conclusively demonstrated that T cell regulation of immunity is an intricate process that involves maintaining self-tolerance while retaining the capacity to mount appropriate immune responses against invading foreign pathogens.

We have previously developed heterologous prime-boost vaccine strategies against a range of pathogens including Plasmodium spp. that cause malaria, Mycobacterium tuberculosis and Hepatitis B Virus. These vaccine strategies are based on the sequential use of different vaccines including the same antigen or epitope of interest such as the use of recombinant protein antigen or plasmid DNA or non-replicating virus vectors that express the antigen of interest. We have found that a number of heterologous prime-boost vaccine strategies are highly efficacious in pre-clinical models as well as in humans.

With respect to malaria vaccines, we have found that a priming vaccination with recombinant Fowlpox9 (FP9), a non-replicating avipoxvirus or recombinant DNA followed by a boosting immunization with recombinant Modified Vaccinia virus Ankara (MVA) is highly efficacious, inducing sterile immunity in the mouse model and in some humans (11-14). Protection against pathogen challenge in the case of malaria (15) and other diseases correlates with the induction of a T cell responses characterised by the production of high levels of IFN-γ.

However, it is apparent that a number of vaccine regimens, including homologous prime-boost immunizations, where the same virus vector based vaccine is used repeatedly, induce a more limited IFN-γ T cell response that is not protective against pathogen challenge. Were it possible to simplify the immunization regimen, to generate for example, a more improved efficacious homologous prime-boost vaccine, this could lead to improved vaccine efficacy in the field. Indeed, the general optimisation of vaccine regimens is very desirable so that they induce sterile immunity in all individuals.

### SUMMARY OF THE INVENTION

According to one aspect of the invention, there is provided a method of inducing an immune response in an organism, comprising the step of administering a vaccine under conditions in which the function of Treg cells is impaired. This novel vaccine approach significantly improves the immune response induced by immunization with vaccine in a non-Treg-depleted environment. The invention has particular utility for inducing cellular immune responses, especially of the CD8+ type.

In devising the present invention, we have examined methods of affecting the Treg population to improve vaccine efficacy. We wished to test whether the induction of an immune response to a vaccine antigen might be improved when the Treg population is temporarily depleted at the time of immunization. Surprisingly, we found that vaccine immunogenicity is significantly increased when the Treg population is depleted.

No previous studies have investigated this phenomenon. The apparent fragility of the aspects of the Treg system studied so far suggested that regulatory T cells would be overwhelmed by powerfully immunogenic viral vectors used as vaccines and that reducing regulatory T cells would thus not influence the immunogenicity of commonly-used non-replicating vectored vaccines. Even more surprising is that this effect, elucidated herein using anti-CD25 antibody to reduce the number of Treg cells, overcomes the natural immunosuppressive effect of this antibody, which acts to inhibit activated T cells (CD25 is the IL-2 receptor that is an activation marker for T cells and thus administering an antibody against CD25 is immunosuppressive). It is thus surprising that anti-CD25, as well as acting on Treg cells, does not reduce the T cell response itself.

This is thus the first demonstration that vaccine immunogenicity is significantly enhanced when strategies that deplete the regulatory T cell compartment are combined with vector, subunit based or attenuated microbial vaccines. Vaccines that are based on whole non-recombinant viruses are specifically disclaimed from the scope of the present invention.

As used herein, the term “Treg cell” is intended to describe the subpopulation of T cells, particularly T helper cells, that may be characterised by cell surface expression of CD4 and CD25 and act to “suppress” effector T cells in vitro and/or in vivo (herein, CD4+CD25+). The role of these cells has been partially investigated previously. For example, a study by Casares et al. focused on the induction of anti-tumour immunity in a Treg depleted host using a peptide specific for the tumour self-antigen as a vaccine (9). However, it was demonstrated that protection against tumour challenge is dependent on the induction of CD4⁺ T cells when Treg are depleted and is independent of immunization with the peptide vaccine. Other studies looking at Treg and vaccines have focused on depleting Treg at the time of a boosting DNA immunization or using replicating vaccinia virus as a tumour therapy (16, 17). In particular, Kursar et al. found that boosting, by intracellular ballistic administration (by gene gun) of plasmid DNA, of a memory CD8+ response could be inhibited by CD4+ T cells and also to a lesser degree by CD25+ cells. However, conventional needle administration of vectored vaccines, which does not lead to direct intracellular administration was not explored.

Two further articles extend our appreciation of the role of Treg cells in preventing activation of autoreactive T cells. For example, Aandahl et al., (18) demonstrate that the magnitude of a CD8+ T cell response to an acute HSV infection is subject to Treg control. The paper concludes that HSV infection leads to activation of Treg function. Suvas et al. (18) also demonstrate that Treg cells play a role in the control of CD8+ T cell mediated immune responses to acute viral infection, but this discovery is reported in the context of Treg cells being activated by a natural chronic viral infection of mice (involving replication of the virus population), and the authors had no idea that non-replicating vectors would induce Tregs that attenuate immune responses. Suvas was studying natural non-recombinant virus populations and monitored the T cell reaction to a major epitope of that virus that is the target of much of the CD8 T cell immune response, rather than studying the immune response to a recombinant antigen inserted into a viral vector. Given that most of the immune response to a recombinant viral vector is against the vector and not against the insert it is most surprising that Treg cells affect immune responses to the recombinant antigen insert.

In contrast, our study demonstrates that impairment of Treg function, such as by Treg depletion, with vector-based or subunit-based vaccines leads to a significant improvement in immunity, particularly to a non-self antigen of an infectious pathogen. The invention further enables enhancement of the immunogenicity and protective efficacy of other types of vaccine such as subunit protein vaccines, by simple extension of the method currently used to incorporate the step of Treg function impairment, such as by depletion. As Treg cells control a great variety of immune responses it is clear that this method may be used to enhance a variety of antigen-specific immune responses including CD8+ T cell responses, CD4+ T cell responses and antibody responses.

The following types of vaccines are illustrated herein:—viral vectors including adenovirus, MVA, FP9 and BCG (an attenuated mycobacterium) used as a vaccine against tuberculosis; a subunit vaccine comprising a protein mixed with an adjuvant; and plasmid DNA. All these types of vaccine may be used in conjunction with the method of the present invention. Other suitable types will be clear to those of skill in the art on reading this specification.

According to the methodology of the invention, the vaccine must be administered to an organism under conditions in which the function of T cells has been impaired. Preferably, an agent is administered to the organism that impairs the function of Treg cells. By “impairs the function of Treg cells”, is meant any method that effectively depletes the function of the regulatory T cell compartment. This may be by depletion of the Treg cells themselves or may be through inactivation of the Treg cells. Monoclonal antibodies that impair the function of CD25+ cells without depletion have been described (Forrest et al. Transplant Immunology 14:43-47, 2005).

Treg cell function is ideally impaired to the maximum degree possible. For example, this may be done by totally depleting the available Treg cell compartment. Preferably, Treg cell function is impaired by at least 25%, more preferably at least 50%, more preferably at least 75%, more preferably at least 90%, more preferably at least 95%, even more preferably 100%. Techniques for measurement of the degree of impairment of function of the Treg cell compartment will be known to those of skill in the art and examples are shown herein.

According to one aspect of the invention, there is provided a method of inducing an immune response in an organism, comprising the step of administering a vaccine, such as a vectored or subunit vaccine, in the presence of an agent that depletes Treg cells, or where the Treg cells in the organism have been depleted. In an alternative but equivalent embodiment, Treg cells are not physically depleted in number, but are depleted (impaired) in function and thus inactivated. This inactivation removes the Treg cell activity and thus has the same effect as Treg cell depletion.

This aspect of the invention also provides the use of an agent that impairs the function of Treg cells to enhance a method of inducing an immune response in an organism, particularly using a vectored vaccine and/or a non-self antigen. The Treg cell function may be impaired through depletion of the Treg cells or by their inactivation.

The invention further provides the use of an agent that impairs the function of Treg cells in the manufacture of a medicament for the treatment or prevention of an infectious disease caused by a pathogen. The Treg cell function may be impaired through depletion of the Treg cells. The pathogen may or may not be a virus.

This effect noted by the inventors is particularly potent in conjunction with prime-boost protocols, such as those described in EP0979284, the contents of which are incorporated herein in their entirety. Accordingly, this aspect of the invention provides a method of inducing an immune response in an organism, comprising the steps of exposing the organism to a priming composition that comprises an antigen in a vectored vaccine, and boosting the immune response by administering a boosting composition comprising a vectored vaccine including the same antigen that was present in the priming composition, wherein the Treg cell function in the organism is impaired, and Treg cells may be depleted, prior to or at substantially the same time as the priming composition is administered.

This aspect of the invention also relates to a method for generating an immune response in a mammal, comprising administering to said mammal at least one dose of each of the following:

i) a priming composition that comprises an antigen in a vectored vaccine;
ii) a boosting composition comprising a vectored vaccine including the same antigen that was present in the priming composition;
wherein the Treg cell function in the organism is impaired, and Treg cells may be depleted prior to or at substantially the same time as the priming composition is administered.

A further aspect of the invention also relates to a method for generating an immune response in a mammal, comprising administering to said mammal at least one dose of each of the following:

i) a priming composition that comprises an antigen in a vectored vaccine;
ii) a boosting composition comprising a vectored vaccine including the same antigen that was present in the priming composition;
wherein an anti-CD25 antibody is administered to the mammal at substantially the same time as the priming composition is administered.

This aspect of the invention also provides for the use of a boosting composition comprising a vectored vaccine, in the manufacture of a medicament for the treatment or prevention of a disease in an organism which has been exposed to a priming composition and that has been depleted of Treg cells.

The invention also provides for the use of a boosting composition comprising a vectored vaccine, in the manufacture of a medicament for the treatment or prevention of a disease in an organism which has been exposed to a priming composition and that has been exposed to an anti-CD25 antibody.

The invention also includes kits, adapted to be used in the vaccination methods described above. An example of such a kit will comprise:

i) an agent that is capable of impairing Treg cell function in an organism, and may deplete Treg cells, when administered to the organism;
ii) a priming composition comprising a source of one or more antigens encoded by a non-replicating or replication-impaired recombinant viral vector, and
iii) a boosting composition comprising a source of one or more antigens encoded by a non-replicating or replication-impaired recombinant viral vector, including at least one antigen which is the same as an antigen of the priming composition.

In another aspect the invention provides a method for generating an immune response against at least one antigen, which method comprises administering at least one dose of component (i), substantially simultaneous with or followed by at least one dose of component (ii), followed by at least one dose of component (iii) of the kit described above.

A still further aspect of the invention provides for the use of an agent that is capable of impairing Treg cell function in an organism, and may deplete Treg cells, when administered to the organism, for the manufacture of a medicament for simultaneous, separate or sequential application with a vectored or subunit vaccine, in order to induce an immune response in an organism against an antigen contained within the vaccine. The vaccine may be a prime boost vaccine. In one embodiment of this aspect the agent may be used for simultaneous, separate or sequential application with a priming composition of a prime boost vaccine.

By “in the presence of an agent that depletes T cells” is meant that the vectored vaccine is administered to the organism subsequent to or at substantially the same time as the agent that depletes Treg cells. The organism need not be permanently depleted of Treg cells, but can be temporarily depleted.

Preferably, the vectored vaccine is a viral vectored vaccine.

Preferably, the vectored vaccine comprises a source of non-self antigen.

The term “vectored vaccines” is well known in the art and includes plasmid DNA, recombinants of poxviruses such as MVA, replicating vaccinia, fowlpox, avipox, also of adenoviruses including non-human primate adenoviruses, of alphaviruses, of vesicular stomatitis virus, and bacterial vectors such as Salmonella, Shigella and BCG.

Preferably, the vectored vaccine contains a recombinant antigen. More preferably, the recombinant antigen is expressed in a viral vector. Even more preferably, the antigen is a non-self antigen.

Examples of viral vectors that are useful in this context are vaccinia virus vectors such as MVA or NYVAC. A preferred viral vector is the vaccinia strain modified virus ankara (MVA) or a strain derived from MVA. Alternatives to vaccinia vectors include avipox vectors such as fowlpox or canarypox vectors. Particularly suitable as an avipox vector is a strain of canarypox known as ALVAC (commercially available as Kanapox), and strains derived from ALVAC.

Preferably, the vector used in the method according to the invention is a non-viral vector or a non-replicating or replication-impaired viral vector. In the case of prime boost protocols, the source of antigen in the priming composition is preferably not the same poxvirus vector or not a poxvirus, so that there is minimal cross-reactivity between the primer and the booster. Further details of preferred protocols for use with prime boost vaccines are disclosed in EP-A0979284. Among other things, this patent application discloses that recombinant MVA and other non-replicating or replication-impaired strains are surprisingly and significantly better than conventional recombinant vaccinia vectors at generating a protective CD8+ T cell response, when administered in a boosting composition following priming with a DNA plasmid, a recombinant Ty-VLP or a recombinant adenovirus.

The term “non-replicating” or “replication-impaired” as used herein means not capable of replication to any significant extent in the majority of normal mammalian cells or normal human cells. Viruses which are non-replicating or replication-impaired may have become so naturally (i.e. they may be isolated as such from nature) or artificially e.g. by breeding in vitro or by genetic manipulation, for example deletion of a gene which is critical for replication. There will generally be one or a few cell types in which the viruses can be grown, such as CEF cells for MVA.

Replication of a virus is generally measured in two ways: 1) DNA synthesis and 2) viral titre. More precisely, the term “non-replicating or replication-impaired” as used herein and as it applies to poxviruses means viruses which satisfy either or both of the following criteria:

1) exhibit a 1 log (10 fold) reduction in DNA synthesis compared to the Copenhagen strain of vaccinia virus in MRC-5 cells (a human cell line);
2) exhibit a 2 log reduction in viral titre in HELA cells (a human cell line) compared to the Copenhagen strain of vaccinia virus.

Examples of poxviruses which fall within this definition are MVA, NYVAC and avipox viruses, while a virus which falls outside the definition is the attenuated vaccinia strain M7.

Alternative preferred viral vectors for use in the priming composition according to the invention include a variety of different viruses, genetically disabled so as to be non-replicating or replication-impaired. Such viruses include for example non-replicating adenoviruses such as El deletion mutants. Genetic disabling of viruses to produce non-replicating or replication-impaired vectors has been widely described in the literature (e.g. McLean et al. 1994).

Other suitable viral vectors for use in the priming composition are vectors based on herpes virus and Venezuelan equine encephalitis virus (VEE) (Davies et al. 1996). Suitable bacterial vectors for priming include recombinant BCG and recombinant Salmonella and Salmonella transformed with plasmid DNA (Daiji A et al. 1997 Cell 91: 765-775).

Alternative suitable non-viral vectors for use in the priming composition include lipid-tailed peptides known as lipopeptides, peptides fused to carrier proteins such as KLH either as fusion proteins or by chemical linkage, antigens modified with a targeting tag, for example C3d or C4b binding protein, whole antigens with adjuvant, and other similar systems. Adjuvants such as QS21 or SBAS2, also known as AS02. (Stoute J A et al. 1997 N Engl J Medicine 226: 86-91) may be used with proteins, peptides or nucleic acids to enhance the induction of T cell responses. These systems are sometimes referred to as “immunogens” rather than “vectors”, but they are vectors herein in the sense that they carry relevant CD8+ T cell epitopes.

There is no reason why the priming and boosting compositions should not be identical in that they may both contain the priming source of antigen and the boosting source of antigen as defined above. A single formulation which can be used as a primer and as a booster will simplify administration.

In the case of CD8+ T cell epitopes either present in, or encoded by the priming and boosting compositions, these may be provided in a variety of different forms, such as a recombinant string of one or two or more epitopes, or in the context of the native target antigen, or a combination of both of these. CD8+ T cell epitopes have been identified and can be found in the literature, for many different diseases. It is possible to design epitope strings to generate a CD8+ T cell response against any chosen antigen that contains such epitopes. Advantageously, the epitopes in a string of multiple epitopes are linked together without intervening sequences so that unnecessary nucleic acid and/or amino acid material is avoided. In addition to the CD8+ T cell epitopes, it may be preferable to include one or more epitopes recognized by T helper cells, to augment the immune response generated by the epitope string. Particularly suitable T helper cell epitopes are ones which are active in individuals of different HLA types, for example T helper epitopes from tetanus (against which most individuals will already be primed). It may also be useful to include B cell epitopes for stimulating B cell responses and antibody production.

In EP-A-0979284, the contents of which are specifically incorporated herein by reference, it was shown that the greatest immunogenicity and protective efficacy is surprisingly observed with non-replicating vectors, rather than replicating vectors that had been used previously. Non-replicating vectors have an added advantage for vaccination in that they are in general safer for use in humans than replicating vectors.

The priming and boosting compositions described may advantageously comprise an adjuvant. In particular, a priming composition comprising a DNA plasmid vector may also comprise granulocyte macrophage-colony stimulating factor (GM-CSF), or a plasmid encoding it or other cytokines, chemokines or growth factors, to act as an adjuvant; beneficial effects are seen using GM-CSF in polypeptide form.

When used in the context of prime boost protocols, the methods of the invention may utilise either homologous or heterologous prime boost immunization regimes.

Specifically, it is reported herein that impairing Treg cell function by depleting Treg cells before a priming immunization with recombinant virus vector vaccines (poxvirus and adenovirus) significantly increases the induction of antigen-specific immunity after a priming immunization (see FIGS. 1a and d), as measured in peripheral blood mononuclear cells (PBMC).

It has also been found that depleting Treg before homologous immunizations, using recombinant MVA, significantly improves vaccine induced immunity as measured in PBMC. Immune responses in spleen and in lymph node are also increased in Treg depleted compared to control animals (FIGS. 1c, 2c and 3). This is observed when the vaccine antigen is of parasite (FIG. 1), bacterial origin (FIG. 3a), viral origin (FIG. 5) or is a string of tumour epitopes (FIG. 3b). Significantly, depleting Treg before immunization with a subunit vaccine of Hepatitis B virus surface antigen (HBsAg) in alum also improves vaccine induced immunity to the antigen (FIG. 5).

It has also been found that depleting Treg before prime-boost heterologous immunization significantly improves vaccine immunogenicity in PBMC, in spleen and in lymph node, compared to normal, non-Treg depleted animals (FIGS. 2 and 4). This has so far been investigated using DNA/MVA, FP/MVA, ADV/MVA and BCG/MVA prime-boost heterologous immunization (FIGS. 2 and 4). These immunisation protocols are examples of preferred protocols according to the invention.

The present invention may be used to enhance a variety of immune responses, as described above. In particular, it is an aim of this invention to identify an effective means of immunizing against diseases in which T cell responses play a protective role. Such diseases include but are not limited to malaria, infection and disease caused by the viruses HIV, herpes simplex, herpes zoster, hepatitis C, hepatitis B, influenza, Epstein-Barr virus, measles, dengue and HTLV-1; by the bacteria Mycobacterium tuberculosis and Listeria sp.; by encapsulated bacteria such as streptococcus and haemophilus; and by parasites such as Leishmania, Toxoplasma and Trypanosoma; and certain forms of cancer e.g. melanoma, lymphomas and leukaemia, cancers of the lung, breast and cancer of the colon.

Various methods of impairing Treg cell function, for example, by depleting Treg cells are known in the art. These cells can be characterised by the expression of CD25 on the cell surface. Impairment of Treg cell function can be achieved by targeting CD25⁺ cells by the use of antibodies, preferably monoclonal antibodies; this is a preferred mechanism to achieve this. Impairment of Treg cell function, for example, by targeting CD25⁺ cells is feasible in clinical practice and is useful particularly for renal transplantation and a good safety record is evident. Two products for human use are available on the market:—basiliximab (Novartis) and daclizumab (Roche).

By “antibodies directed against CD25” is meant that such antibodies display strong binding affinity for CD25. The antibodies directed against CD25 may directed to the cell surface CD25 or soluble CD25 or may be directed to both these forms of the protein. The antibodies will preferably be immunospecific for CD25. The term “immunospecific” means that the antibodies have substantially greater affinity for CD25 than their affinity for other related polypeptides in the prior art. By “substantially greater affinity” we mean that there is a measurable increase in the affinity for CD25 as compared with the affinity for other related proteins. Preferably, the affinity is at least 10-fold, 100-fold, 10³-fold, 10⁴-fold, 10⁵-fold or 10⁶-fold greater for CD25 than for other related proteins.

In addition to the use of anti-CD25 antibodies, Treg cell function may be impaired through the use of fragments of anti-CD25 antibodies such as Fv components or by camelids. Other possibilities are provided by the use of compounds that disrupt the interaction between CD25 and the ligand IL-2. Treg cells require IL2 for their activity and survival, so blocking this interaction has the effect of impairing the function of Treg cells. Accordingly, proteins such as IL2, or proteins in which IL2 is fused to a toxin such as diphtheria toxin, may be used, as well as antibodies directed to CTLA-4 or GITR and other markers expressed on Treg cells. One such example is LMB-2, a recombinant immunotoxin consisting of a single-chain Fv fragment of the anti-CD25 monoclonal antibody fused to Pseudomonas exotoxin, that is in clinical trials. Still further possibilities will be apparent to the skilled reader, and include methods that are subsequently devised as research in this area progresses.

One aspect of the invention is therefore a method of inducing an immune response in an organism, comprising the step of administering a vaccine in the presence of an agent that disrupts the interaction between CD25 (IL-2R alpha) and one or more of its ligands. The agent may disrupt the interaction between cell surface CD25 and one or more of its ligands, between soluble CD25 and one or more of its ligands or between both cell surface and soluble CD25 and one or more of its ligands. An example of a ligand of CD25 is IL-2. Agents that are suitable for use in this aspect of the present invention include any agent that reduces the effective concentration of a ligand for CD25. Examples of such agents include soluble CD25 (which binds to IL-2). This mode of action works by mopping up IL-2 and starving Treg cells of this cytokine that these cells require for their growth (see Zorn et al., Cytokine. 1994 July; 6(4):358-64 “Soluble interleukin 2 receptors abrogate IL-2 induced activation of peripheral mononuclear cells.”; Gooding et al., Br J Cancer. 1995 August; 72(2):452-5 “Increased soluble interleukin-2 receptor concentration in plasma predicts a decreased cellular response to IL-2”). Another agent suitable for use in this aspect of the invention are those that bind to and block inhibitory soluble CD25. Proteins such as IL2, or proteins in which IL2 is fused to a toxin such as diphtheria toxin, may be used. Another example of an agent that disrupts the interaction between CD25 and one of its ligands is an agent that binds to CD25 and which therefore blocks the interaction between IL-2 and its receptor CD25. A preferred example of this type of agent is anti-CD25 antibody, examples of which include basiliximab (Novartis) and daclizumab (Roche). Further examples might include agents that down-regulate the activity or levels of CD25, including agents that reduce the level of transcription of CD25, reduce the level of translation of CD25 (examples of which include antisense RNA and siRNA), reduce the amount of CD25 that reaches the cell membrane, reduce the activity of CD25 protein (such as suicide inhibitors) and so on. Other examples will be clear to those of skill in the art.

All such methods of this aspect of the invention, that target the interaction of CD25 with one or more of its ligands, either may or may not result in the impairment of Treg function, such as by depletion of T cells.

One aspect of the invention is therefore a method of inducing an immune response in an organism, comprising the step of administering a vaccine in the presence of an agent that binds to CD25. Such an agent may be an anti-CD25 antibody. Suitable anti-CD25 antibodies may bind to cell surface CD25 and/or soluble CD25. Methods according to this aspect of the invention either may or may not result in the impairment of Treg function, such as by depletion of T cells. This aspect of the invention also provides the use of an anti-CD25 antibody to enhance a method of inducing an immune response in an organism, particularly using a vectored vaccine and/or a non-self antigen. The invention further provides the use of an anti-CD25 antibody in the manufacture of a medicament for the treatment or prevention of an infectious disease caused by a pathogen. The pathogen may or may not be a virus. A still further aspect of the invention provides for the use of an anti-CD25 antibody for the manufacture of a medicament for simultaneous, separate or sequential application with a vectored or subunit vaccine, in order to induce an immune response in an organism against an antigen contained within the vaccine.

Methods of impairing Treg cell function might include for example, the use of agents that target molecules that are important to the function of Treg cells. Examples of molecules important to the function of Treg cells include CTLA-4 (cytotoxic T-lymphocyte-associated protein 4), GITR (tumour necrosis factor receptor superfamily, member 18), IL-10 (interleukin 10), TGF-beta or FoxP3 (forkhead box P3). For example, suitable agents are antibodies or ligands or other molecules that interact with these molecules, or that down-regulate their activity, level or function in some other way. Examples of suitable agents are antibodies against CTLA-4, GITR, IL-10, TGF-β or FoxP3. Other examples might include siRNA—for example, siRNA to FoxP3 could be used to silence FoxP3 and allow the T cells to regain effector cell function. Still further possibilities will be apparent to the skilled reader, and include methods that are subsequently devised as research in this area progresses.

All such agents share a mechanism of action in common, that results in the enhancement of vaccine-induced immunity. The vaccine may be a vectored vaccine or a subunit vaccine.

The timing of the impairment of Treg cell function, or depletion of the Treg cells themselves, and/or administration of anti-CD25 antibody is also of interest. According to the invention, vaccine components should be administered in the presence of an agent that impairs Treg cell function, such as by depleting Treg cells and/or an anti-CD25 antibody. By this is meant that a component of the vaccine should be administered either substantially simultaneously with, or after the administration of the agent.

For example, it is specifically shown herein that administering vaccine a number of days after Treg depletion significantly improves the immune response induced by vaccine alone (see FIG. 1). The vaccine may be administered 1, 2, 3, 4, 5, 6, 7, 8, 9, 10, 15 or 20 or more days after the agent that impairs Treg cell function, e.g. depletes Treg cells. Preferably, the vaccine may be administered around 5 days after the agent. In an alternative preferred embodiment, the vaccine may be administered less than around 70 hours after the agent, particularly in the case of an anti-CD25 antibody. As the skilled reader will be aware, the precise timing of administration of agent will depend on the type of agent used and the amount of time necessary for this agent to impair Treg cell function appropriately.

In the case of prime boost immunisation regimes, the agent that impairs the function of Treg cells is preferably administered at substantially the same time as the priming composition, or alternatively before the priming composition. As above, the priming composition may be administered 1, 2, 3, 4, 5, 6, 7, 8, 9, 10, 15 or 20 or more days after the agent that impairs Treg cell function. Preferably, the priming composition may administered around 5 days after the agent that impairs Treg cell function. The agent that impairs Treg cell function may be administered on one or more occasion. In an alternative preferred embodiment, the vaccine may be administered less than around 70 hours after the agent that impairs Treg cell function, particularly in the case of an anti-CD25 antibody. It is specifically reported herein that co-administering the Treg depleting agent with the vaccine, in the same syringe, significantly improves the immune response to the vaccine antigen after one immunization and after homologous prime-boost immunization. FIGS. 1, 3 and 8 show co-administration of anti-CD25 antibody with MVA, as tested in PBMC and spleen. Co-administering the Treg depleting agent with the vaccine also results in broader and more durable immune responses (FIGS. 6 and 7). The breadth of the immune response, that is, the number of epitopes recognised by T cells is of interest as a T cell response to a broad range of epitopes may reduce or avoid selection of pathogen escape mutants and may provide increased protection against viral or parasite challenge. More durable immunity results in more effective and more long-lasting protection against pathogen challenge subsequent to immunization.

It has also been found that administering vaccine at the same time as impairing Treg cell function, such as by depleting Treg, at a different site significantly improves vaccine efficacy after a single immunization (FIG. 1d). Administering vaccine at the same time as depleting Treg at a different site also significantly improves vaccine efficacy after homologous prime-boost immunization (FIGS. 1, 3 and 5).

The invention is applicable to a variety of different organisms, including for example, vertebrates, large animals and primates. Although medical applications with humans are clearly foreseen, veterinary applications are also envisaged here.

The compositions and methods described herein may be employed as therapeutic or prophylactic vaccines. Whether prophylactic or therapeutic immunization is the more appropriate will usually depend upon the nature of the disease. For example, it is anticipated that cancer will be immunized against therapeutically rather than before it has been diagnosed, while anti-malaria vaccines will preferably, though not necessarily be used as a prophylactic. CD8+ T cell responses are well known to be of particular value in immunotherapy.

The compositions according to the invention may be administered via a variety of different routes. Certain routes may be favoured for certain compositions, as resulting in the generation of a more effective response, or as being less likely to induce side effects, or as being easier for administration.

For example, the compositions utilised in this invention may be administered by any number of routes including, but not limited to, oral, intravenous, intramuscular, intra-arterial, intramedullary, intrathecal, intraventricular, transdermal or transcutaneous applications, subcutaneous, intraperitoneal, intranasal, enteral, topical, sublingual, intravaginal or rectal means. Gene guns or hyposprays may also be used to administer the compositions of the invention. Typically, the compositions may be prepared as injectables, either as liquid solutions or suspensions; solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. Dosage treatment may be a single dose schedule or a multiple dose schedule.

The invention may prove to be particularly useful in cancer immunotherapy where administration of monoclonal antibodies to CD25 and therapeutic vaccines would be particularly practicable. Several target antigens and epitopes for cancer immunotherapy are well known in the field, for example, MAGE, BAGE, tyrosinase, NY-ESO, MUC-1 and HER2neu; and many CD8 T cell epitopes for particular HLA types are defined in such antigens.

Various aspects and embodiments of the present invention will now be described in more detail by way of example.

It will be appreciated that modification of detail may be made without departing from the scope of the invention.

### BRIEF DESCRIPTION OF THE FIGURES

FIG. 1. Anti-CD25 antibody enhances immune responses induced by homologous recombinant virus-vector prime-boost immunization.

Anti-CD25 antibody was administered by an i.p. route the day prior to and on the day of immunization with MVA-CSP (a-c), or with FP9, DNA or adenovirus expressing PbCSP (FP-CSP or ADV-CSP respectively) (d-h), or it was mixed with MVA-CSP and co-administered by the i.d. route (a-c). A second injection of 0.5 mg anti-CD25 was given by the i.d. route to the latter group on day +1. Control littermates were immunized with the corresponding vaccine. CD8⁺ T cell IFN-γ responses to the dominant Pb9 epitope were assessed in PBMC on day 10 post-vaccination (a, d, g). Mice were boosted with the same vectored vaccine on day 14. T cell responses in the blood were measured 10 days after this boost (b, e), by elispot. T cell responses to Pb9 were assessed in the spleen 2 weeks post-boost (c, f, h). Columns represent the mean number of IFN-γ spot forming cells (SFC) per million splenocytes ±SEM (n=14 to 30 per group in all groups, except DNA immunized groups where n=3). *, P<0.05, ***, P<0.001 compared to antibody untreated vaccinated mice. Similar results were obtained in five independent experiments.

FIG. 2. Treg depletion enhances immune responses induced by heterologous DNA/MVA, FP/MVA or ADV/MVA immunization.

Anti-CD25 antibody was administered by an i.p. route 10 and 8 or the day prior to and on the day of immunization with DNA-CSP, FP-CSP or ADV-CSP. No difference was observed when anti-CD25 was administered at these different times. All mice were boosted on day 14 with MVA-CSP and CD8⁺ T cell responses to Pb9 were assessed in peripheral blood 10 days later (a), or in the spleen (b), or lymph node (c) 2 weeks post-boost, by elispot. Columns represent the mean number of IFN-γ spot forming cells (SFC) per million splenocytes ±SEM (n=8 for FP-CSP groups and n=4 for DNA-CSP and ADV-CSP groups). *, P<0.05, ***, P<0.001 compared to antibody untreated mice that were immunized with the same vaccine regime. Cervical lymph nodes, which drain the intradermal injection site, from individual mice were pooled within groups. Similar results were obtained in two independent experiments.

FIG. 3. Anti-CD25 antibody enhances CD4⁺ T cell responses to M. tb and CD8⁺ T cell responses to tumor epitopes.

Anti-CD25 antibody was administered by an i.p. route two days prior to and on the day of immunization with MVA expressing M. tb Antigen 85A (MVA85A) (a) or expressing a string of tumor epitopes (MVA-T) (b). Antibody treated or control mice received a homologous boost on day 14. CD4+ T cell IFN-γ responses to the MHC class II restricted epitope of Ag85A, termed p15 of the amino acid sequence; TFLTSELPGWLQANRHVKPT, were assessed in PBMC two weeks after the final vaccine (a). CD8⁺ T cell IFN-γ responses to the MHC class I restricted epitopes from P815, LPYLGWLVF or to the CT26 gp70 epitope, SPSYVHQF, were assessed in PBMC from mice immunized with MVA-T two weeks after the homologous boost (b), by elispot. Columns represent the mean number of IFN-γ spot forming cells (SFC) per million splenocytes ±SEM (n=6 per group). *, P<0.05, compared to antibody untreated mice that were immunized twice with MVA85A or MVA-T. Similar results were obtained in three independent experiments.

FIG. 4. Treg depletion enhances T cell responses induced by a bacterial based vaccine.

Anti-CD25 antibody was administered by an i.p. route the day prior to and on the day of immunization with BCG. These mice or control, untreated but vaccinated mice, were boosted with MVA85A four weeks after this priming immunization. CD4+ T cell IFN-γ responses to the p15 epitope were assessed in spleen two weeks post-boost. Columns represent the mean number of IFN-γ spot forming cells (SFC) per million splenocytes ±SEM (n=4 per group). *, P<0.05, compared to antibody untreated vaccinated mice. Similar results were obtained in four independent experiments.

FIG. 5. Treg depletion enhances T cell responses induced by a recombinant subunit protein vaccine.

Anti-CD25 antibody was administered by an i.p. route two days prior to and on the day of subcutaneous (s.c.) immunization with Hepatitis B virus surface antigen (HBsAg) in alum (Engerix-B, GSK, Rixensart, Belgium). These mice or control, antibody untreated vaccinated mice, were boosted with Engerix-B two weeks after the first immunization. CD8⁺ T cell IFN-γ responses to the MHC class I restricted epitope IPQSLDSWWTSL were assessed in spleen two weeks post-boost. Columns represent the mean number of IFN-γ spot forming cells (SFC) per million splenocytes ±SEM (n=5 per group). *, P<0.05 compared to antibody untreated vaccinated mice.

FIG. 6. Treg depletion treatment increases T cell response induced by vaccination to sub-dominant epitopes.

Anti-CD25 antibody was administered by the i.p. route prior to MVA-CSP immunization or was co-administered with MVA-CSP and injected by the i.d. route. Antibody treated or control mice were boosted with MVA-CSP on day 14. Circulating T cell responses in PBMC to the entire PbCSP protein were assessed two weeks after the second immunization using 6 pools of 15mer peptides overlapping by 10 amino acids. Responses to individual pools are shown. Columns represent the mean number of IFN-γ spot forming cells (SFC) per million splenocytes ±SEM (n=5 per group). **, P≦0.005 compared to antibody untreated vaccinated mice. Similar results were obtained in three independent experiments.

FIG. 7. Treg depletion significantly enhances the duration of the immune response.

Mice were injected with 1 mg of anti-CD25 antibody, either by the i.p. route or co-formulated with the MVA-CSP or FP-CSP priming vaccine. CD8⁺ T cell responses to Pb9 in peripheral blood were measured on day 12. All animals were boosted with MVA-CSP on day 14. T cell responses were assessed on day 28, 42, 69 and 100. Points represent the mean number of IFN-γ spot forming cells (SFC) per million splenocytes ±SEM (n=4 per group).

FIG. 8. Low dose anti-CD25 antibody enhances immune responses induced by recombinant virus-vector prime-boost immunization.

Various doses of anti-CD25 antibody were mixed with MVA-CSP and co-administered by the id route. The amount of anti-CD25 antibody used was 1000 μg, 500 μg, 100 μg, 10 μg, 1 μg, 0.5 μg or 0.1 μg. One group of animals were injected with 1 μg anti-CD25 by the i.p. route prior to MVA-CSP immunization. Antibody treated or control mice were boosted with MVA-CSP on day 14. T cell responses in the blood were measured 2 weeks after this boost by elispot. Columns represent the mean number of IFN-γ spot forming cells (SFC) per million splenocytes ±SEM (n=4 per group).

### EXAMPLES

### Example 1

### Treg Depletion in Combination with One Immunisation with Recombinant Virus Vector Based Vaccines

Female Balb/c mice (6 weeks old) were administered 1 mg/mouse anti-CD25 (clone PC61) with a one day interval (0.5 mg/timepoint) at day −7 and −5 or days −3 and −1 day pre-immunisation. Jones et al., (7) have demonstrated that the anti-CD25 Ab is undetectable in the serum after 19 days and that the CD25+ Treg population is fully depleted for up to 10 to 12 days post-antibody administration and that this population slowly returns to 100%, which occurs between day 21 and 29 post-depletion (7). Non-depleted mice were also vaccinated. The vaccine antigen used was Plasmodium berghei circumsporozoite protein (CSP).

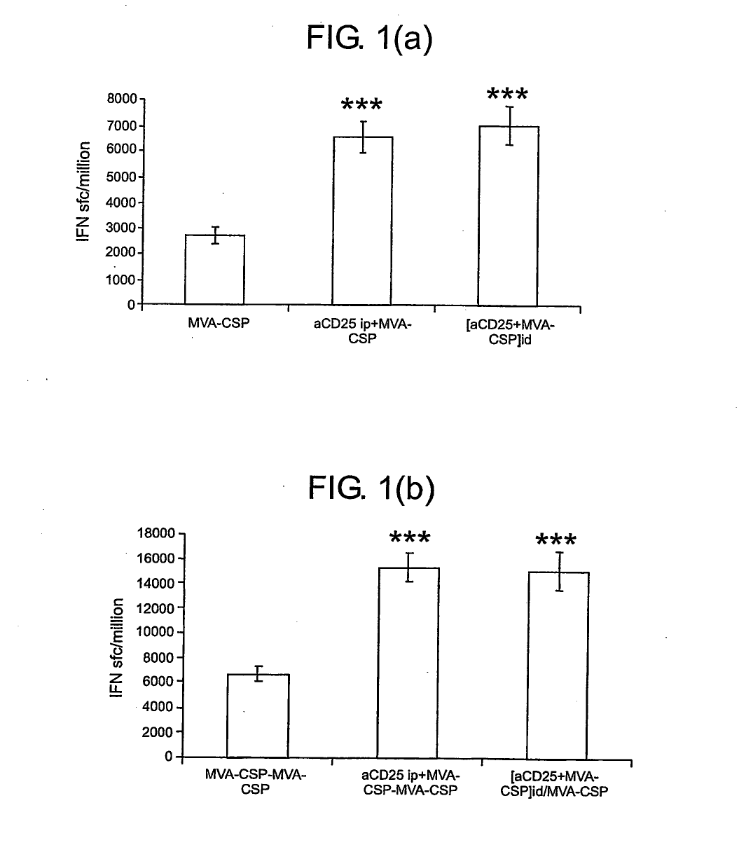

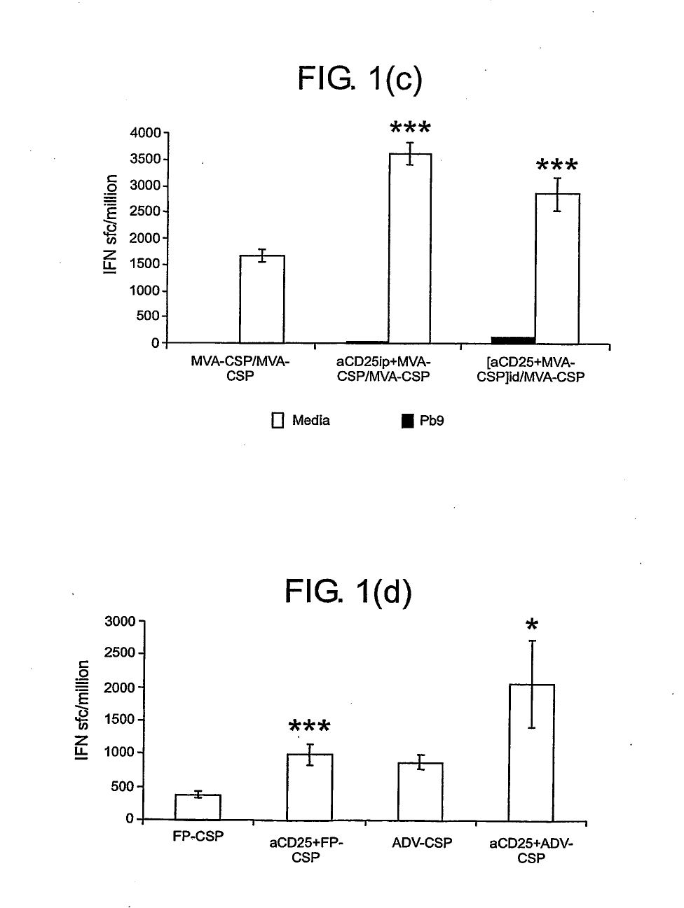

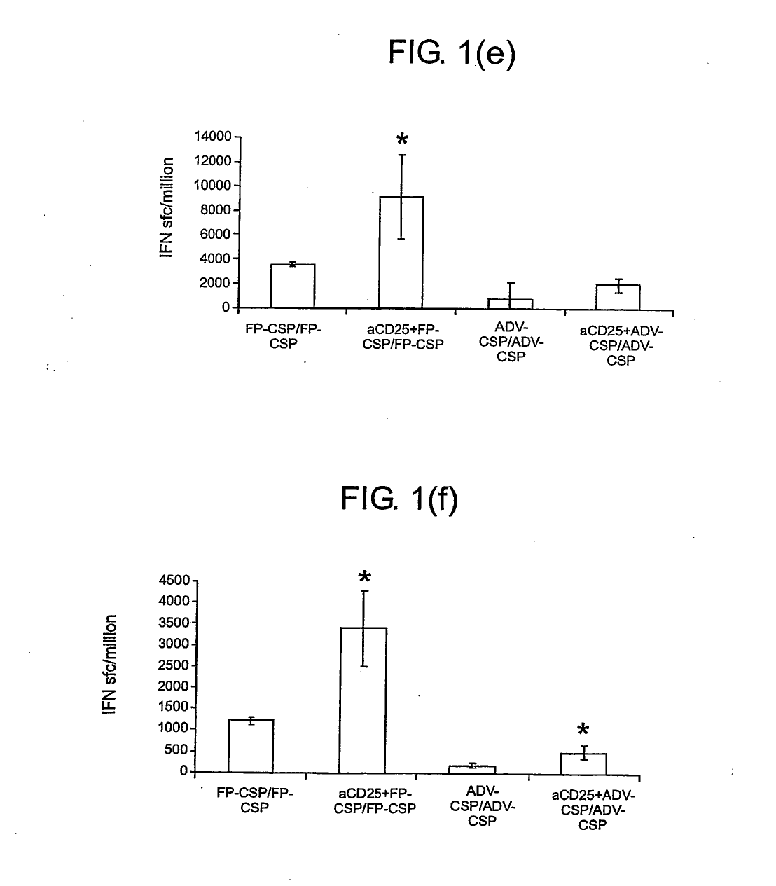

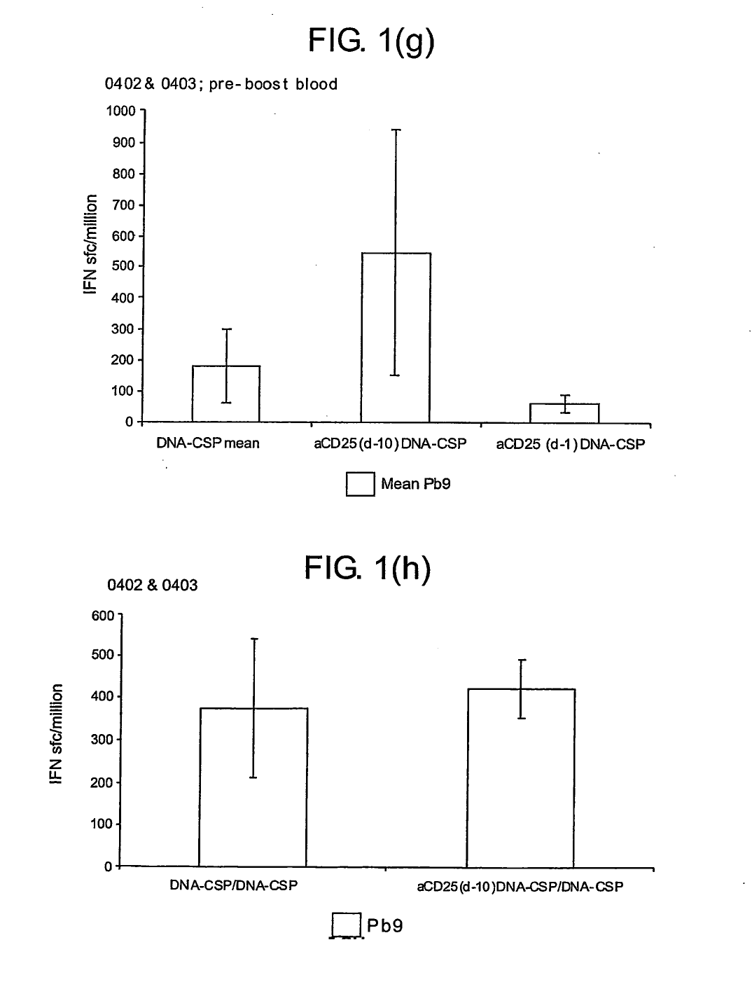

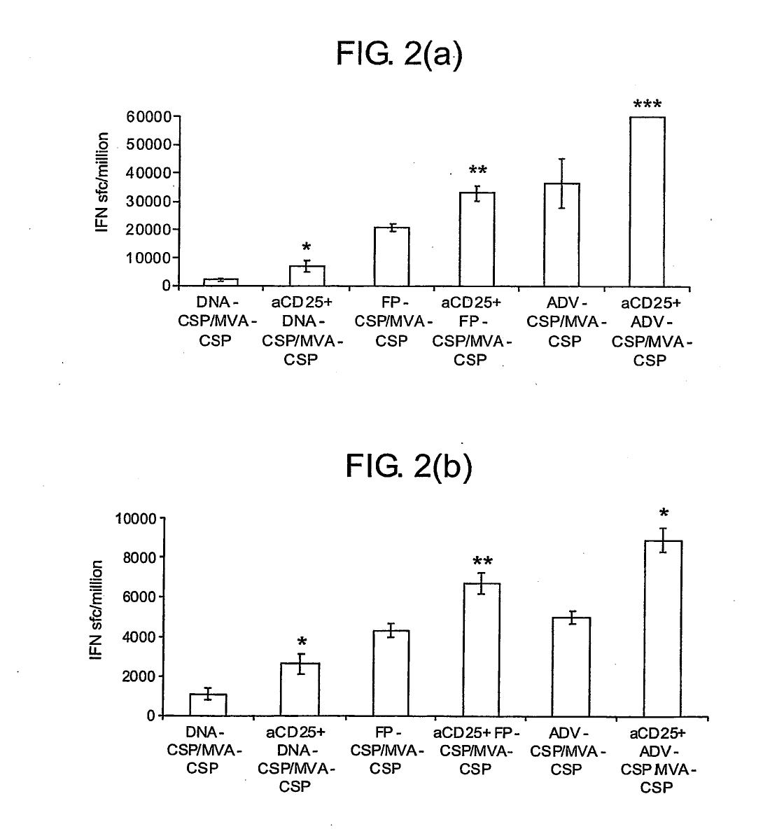

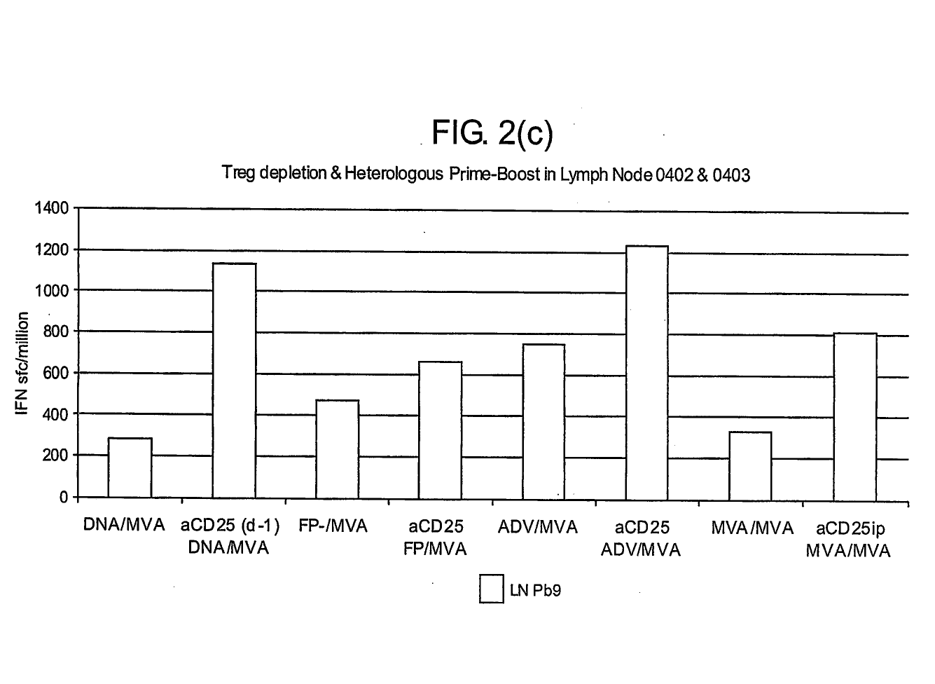

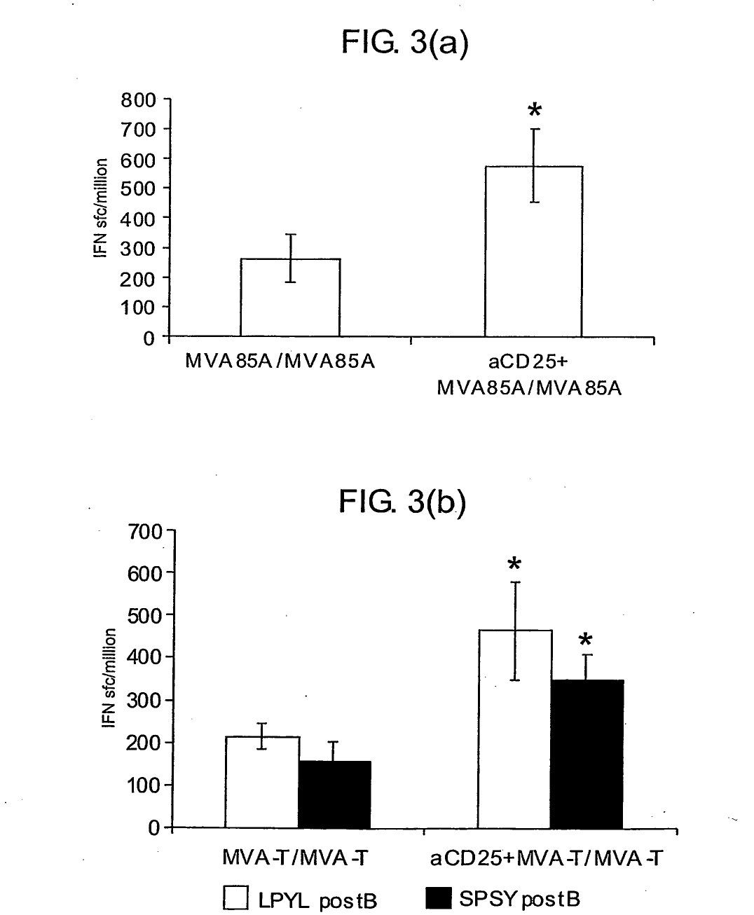

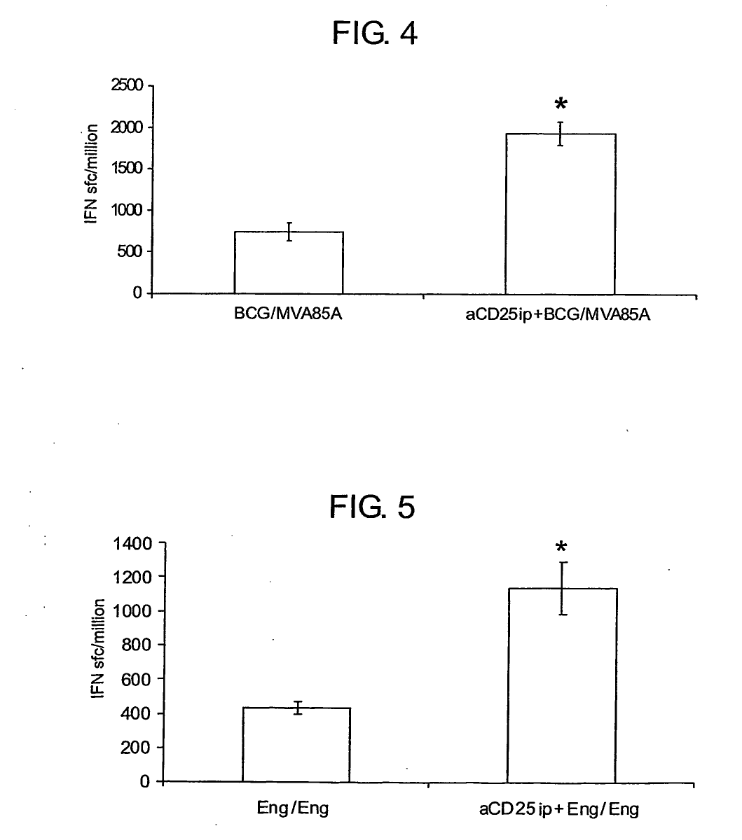

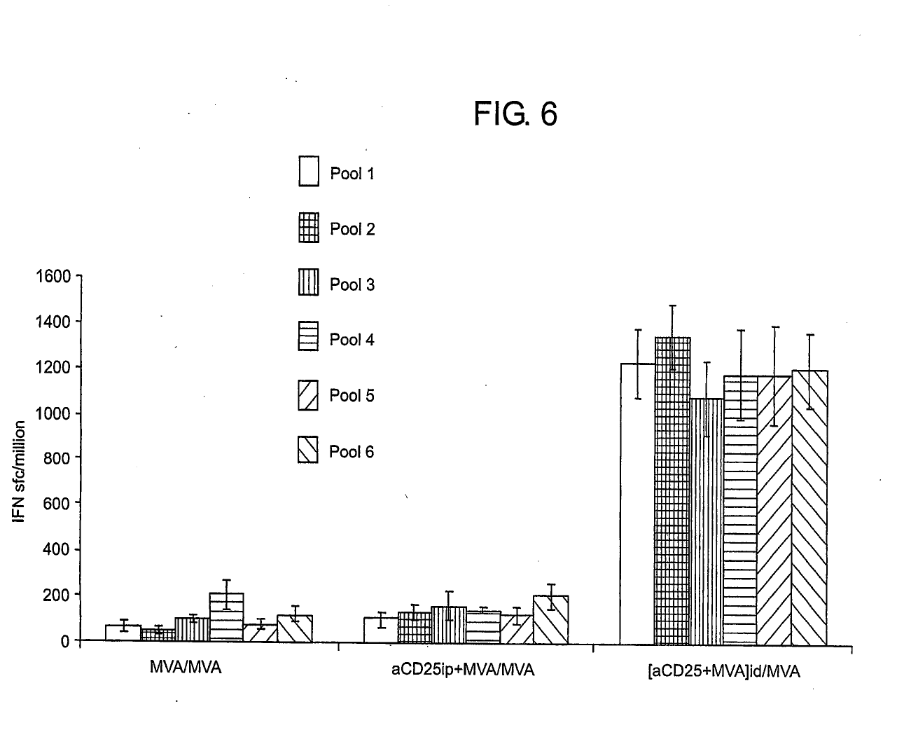

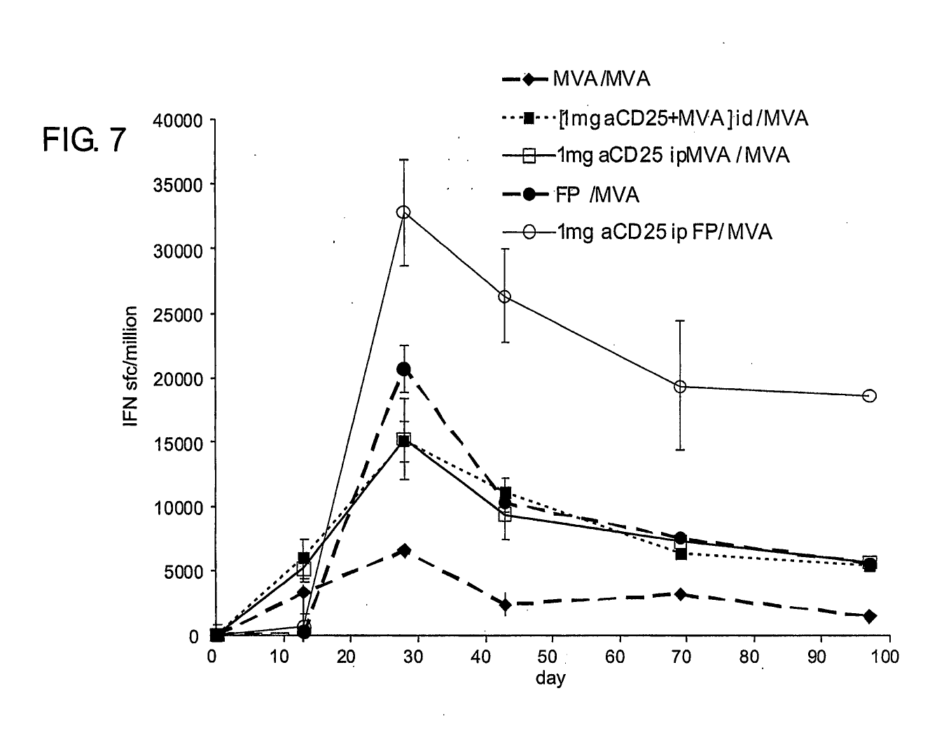

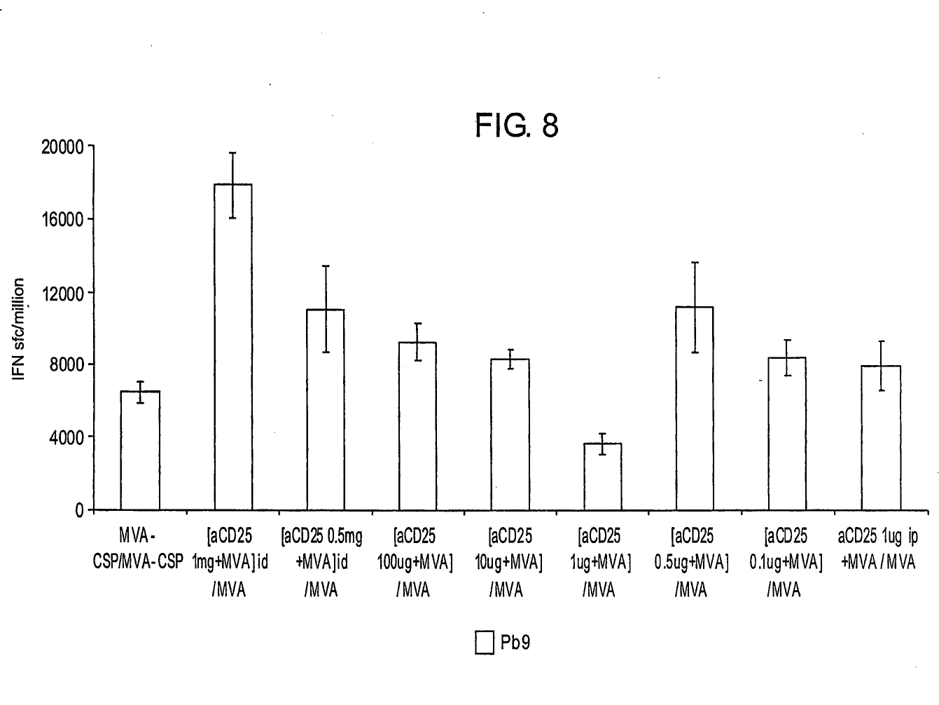

Mice were immunised intradermally (i.d.) with 1×10⁶ pfu recombinant Modified Vaccinia Ankara (MVA-CSP) or Fowlpox9 (FP-CSP) or 1×10⁷ pfu recombinant adenovirus (ADV-CSP) on day 0. PBMC were obtained by tail vein bleeds 10 days post-immunisation. Antigen-specific immune responses were assessed by IFN-γ elispot, using the H-2K restricted epitope of PbCSP, SYIPSAEKI (termed Pb9) using a previously published protocol (11). The circulating antigen-specific CD8+ response induced by one immunisation is a typical response seen in the peripheral blood at this time. FIGS. 1a and 1d demonstrate that Treg depletion significantly enhances the priming of an antigen-specific immune response by a recombinant non-replicating poxvirus (MVA-CSP and FP-CSP) or adenovirus vaccines by three or four-fold the response observed in control, non-depleted mice.

### Example 2

### Treg Depletion in Combination with one Immunisation with DNA-CSP Vaccines may Improve the Immune Response Observed in Blood

As described above, female Balb/c mice (6 weeks old) were administered 1 mg/mouse anti-CD25 (clone PC61) with a one day interval (0.5 mg/timepoint) (7) at day −12 and −10 or days −3 and −1 day pre-immunization. Mice were then immunised intramuscularly (i.m.) with 50 μg DNA-CSP. PBMC were obtained by tail vein bleeds 10 days post-immunisation. Antigen-specific immune responses were assessed by IFN-γ elispot, using the H-2K^{d} restricted epitope of PbCSP, SYIPSAEKI (termed Pb9) using a previously published protocol (11).

As can be observed from FIG. 1g, one intramuscular immunisation with DNA-CSP induces a weak immune response in peripheral blood 10 days after immunisation. Depleting Treg pre-immunisation increases this peripheral blood IFN-γ response. Treg depletion has only a minor effect on the induction of immune responses by two DNA-CSP immunisations. We cannot exclude at this stage that administering anti-CD25 10 days preimmunisation may have affected the induction of this response and as mentioned previously, larger group numbers of DNA immunised mice will be required to increase the statistical power for this particular vaccine strategy.

### Example 3

### Concurrent Treg Depletion and Vaccination Induces a Greater Immune Response Compared to Vaccination Alone

A single shot vaccine where all components of the vaccine are delivered at the same time in the same formulation would be advantageous to improving vaccine efficacy in the field. Alternatively, administering two formulations at the same time in two different sites may be more favourable than delivering these formulations on different days. We examined the effects of co-delivering the depleting Ab on the same day as MVA-CSP priming, either mixed in the same syringe or administered at separate sites. Mice were administered anti-CD25 at the same time as i.d. immunisation at a distal, intraperitoneal site (group labelled “aCD25 i.p.+MVA-CSP”) or the MVA-CSP was mixed with the Treg depleting antibody and this combined formulation was delivered in the same needle intradermally (labelled “[aCD25+MVA-CSP]id”) (FIGS. 1a, 1b and 1c). It can be seen from FIG. 1a that this co-administered formulation leads to greater than 2-fold increase in the primary vaccine induced immune response in peripheral blood, compared to non-antibody-depleted control vaccinated animals. Therefore, local, co-delivery of Treg depleting Ab with a virus vector based vaccine is effective at increasing the immune response. Depleting Treg a day before vaccination is not significantly different to depleting Treg at the same time as vaccination.

This demonstrates that administering vaccine at the same time as depleting Treg at a different site significantly improves vaccine efficacy after a single immunisation.

### Example 4

### Treg Depletion Improves the Responses Induced by Homologous Prime-Boost Immunisation

Treg depletion resulted in an increase peripheral blood T cell responses induced by MVACSP/MVA-CSP by 3-fold compared to control vaccinated mice (FIG. 1b). This significant increase in the post-boost peripheral blood T cell response was achieved when the anti-CD25 was administered 3 and 1 day pre-immunisation, but more interestingly, when the Ab was co-administered in the vaccine formulation or at the same time but at a distal site to the vaccine. Up to this point, inducing this level of immunogenicity has been unattainable with homologous prime-boost immunisation.

When antigen-specific immunity was assessed in spleens and lymph nodes of mice that were immunised twice with MVA-CSP, strong immune responses were observed when the anti-CD25 was injected i.p. 3 and 1 days before immunisation. However, administering the Treg depleting Ab at the same time or at the same place as the vaccine did result in a significant increase in the immune response in the spleen (FIG. 1c, 2c).

### Example 5

### Treg Depletion Combined with Prime-Boost Heterologous Vaccines Induces a Potent Immune Response to the Vaccine Antigen in Peripheral Blood, Spleen and Lymph Node

Control or Treg depleted animals that were immunised with DNA-CSP, FP-CSP or ADV-CSP were boosted with MVA-CSP on day 14 post-prime (FIG. 2). Peripheral blood was monitored 10 days post-boost and immune responses in the spleen and lymph node were examined 14 days post-boost. Depleting Treg resulted in a 3-fold increase in Pb9 responses in peripheral blood lymphocytes when mice were prime-boost vaccinated with DNA-CSP/MVA-CSP. Peripheral blood responses induced by FP-CSP/MVA-CSP and ADV-CSP/MVA-CSP were improved 2-fold when Tregs were absent compared to control non-depleted animals (FIG. 2a).

We also observed a greater than 2-fold increase in the antigen-specific immune response induced by DNA-CSP/MVA-CSP and ADV-CSP/MVA-CSP immunisation in the spleen of Treg depleted animals (FIG. 2b). The Pb9 spleen responses induced by FP-CSP/MVA-CSP were significantly increased when aCD25 was used to deplete Treg. This is novel, as we have been previously unable to further improve the immunogenicity observed in the spleen after heterologous FP/MVA prime-boost vaccination using various other vaccine strategies.

The magnitude of the enhancement of the immune response was enhanced in lymph nodes of depleted and FP-CSP/MVA-CSP and ADV-CSP/MVA-CSP vaccinated mice compared to control, undepleted mice (FIG. 2c). Treg depletion resulted in a 5-fold increase in responses in the lymph nodes of mice that were immunised with DNACSP/MVA-CSP. The great magnitude of the immune response in lymph nodes observed here has not been previously observed after DNA/MVA immunisation.

### Example 6

### Treg Depletion Improves CD4⁺ T Cell Responses Induced by Prime-Boost Immunisation to MHC class II Epitopes and CD8⁺ T Cell Responses to Tumour Epitope Strings

We used MVA expressing M. tuberculosis antigen 85A (MVA85A) to determine if anti-CD25 administration had an effect on CD4⁺ T cell responses to MHC class II restricted epitopes, such as the Ag85A epitope, p15. Anti-CD25 antibody administration before MVA85A immunization resulted in a significant increase in the frequency of circulating p15-specific CD4⁺ T cells compared to the control immunized group (FIG. 3a). Furthermore, Treg depletion one day before an immunization with MVA-T, that expresses a string of MHC class I restricted tumor epitopes, resulted in significant increases in the circulating CD8⁺ T cell responses to the CT26 gp70 epitope (SPSYVHQF) and to the P815-specific epitope (LPYLGWLVF) when responses were examined two weeks after a homologous boost with MVA-T (FIG. 3b).

### Example 7

### Treg Depletion Improves Immune Responses Induced by Prime-Boost Immunisation with Bacterial Based Vaccines

To examine if the effects of anti-CD25 treatment was specific to virus vector based vaccines, we immunized mice depleted of Treg with the M. tuberculosis vaccine, BCG. As the kinetics of the induction of immune responses to antigen 85A following BCG vaccination are slower than responses induced by MVA-85A, BCG immunized animals were boosted with MVA-85A four weeks, instead of two weeks, after priming. Mice that were depleted of CD25⁺ cells displayed an almost three-fold increase in the levels of MHC class II restricted CD4⁺ T cells, specific for the p15 peptide, compared to control immunized mice (FIG. 4). This demonstrates that anti-CD25 treatment enhances both CD4⁺ and CD8⁺ T cell responses to antigens.

### Example 8

### Treg Depletion Enhances T Cell Responses Induced by a Recombinant Subunit Protein Vaccine

To examine if the augmentation by Treg depletion is restricted to live or vectored vaccines, we assessed the immune response induced when a licensed recombinant subunit hepatitis B virus vaccine, Engerix-B (GSK Biologicals, Rixensart, Belgium), was administered to mice in the presence or absence of anti-CD25 antibody. Administration of anti-CD25 before immunization with Engerix-B, which is composed of the Hepatitis B virus surface antigen formulated with alum, significantly increased the MHC class I restricted response (FIG. 5). This demonstrates that the enhancing effect of anti-CD25 treatment also appears to work on immune response to foreign antigens through a mechanism that is independent of viral or bacterial infection and stimulation of known toll-like receptors.

### Example 9

### Treg Depletion Enhances the T Cell Response Induced by Vaccination to Sub-Dominant Epitopes

Using pools of overlapping peptides spanning the entire sequence of PbCSP, but omitting the peptide containing the dominant Pb9 epitope, we examined the breadth of the immune response induced by different prime-boost vaccine regimes in the presence or absence of anti-CD25 (FIG. 6). Co-administration of anti-CD25 with MVA-CSP in the priming vaccination significantly increased the T cell responses to subdominant epitopes present in all peptide pools (FIG. 6).

### Example 10

### Treg Depletion Maintains the Effector CD8⁺ T Cell Response Induced by Vaccination at a Higher Level Over Time Compared to Untreated Homologous or Heterologous Prime Boost Vaccination

We examined the immune response induced by combining anti-CD25 treatment with homologous MVA-CSP or heterologous FP/MVA-CSP immunization over the course of 100 days (FIG. 7). We assessed the effect of co-administering the antibody with the vaccine, compared to giving it by the i.p. route on the maintenance of the immune response. The peak of the immune response in the periphery was observed when responses were examined 28 days after priming (FIG. 7). Treatment with anti-CD25 antibody significantly increased all vaccine-induced responses at 28 and 42 days after the priming immunization compared to antibody untreated, vaccinated controls. Analysis of circulating CD8⁺ T cell responses in peripheral blood at day 69 and 100, when the immune system is fully replete after antibody administration, demonstrated that initial Treg depletion in combination with FP-CSP/MVA-CSP induced the strongest response that was sustained. Overall, the results demonstrate that addition of anti-CD25 antibody, to deplete Treg, to the priming vaccine not only enhances the immunogenicity at the peak time-point, but it also augments the durability of this increased response

### Example 11

### Treg Depletion in Conjunction with Vaccination Improves Protective Efficacy Against a Malaria Challenge

To assess whether the augmented immunogenicity observed by Treg depletion and immunization resulted in increased protective efficacy, anti-CD25 antibody treated or untreated mice that were primed with FP-CSP and boosted with MVA-CSP immunization were challenged with P. berghei sporozoites (Table 1). A significant increase in protection was observed when mice received anti-CD25 at the time of FP/MVA vaccination compared to antibody untreated immunized mice (Table 1). The enhanced durability of immunity induced by anti-CD25 and FP/MVA (FIG. 7) is consistent with the increased protection observed against parasite challenge at days 42 and 52.

### Example 12

### Low Dose of Anti-CD25 Treatment Enhances Vaccine-Induced Immune Responses

Varying doses of anti-CD25 antibody were co-administered with a priming vaccination of MVA-CSP. One group of mice received 1 μg anti-CD25 by the i.p. route (FIG. 8). Enhanced CD8+ T cell responses were generated in mice receiving anti-CD25, even at low doses (0.5 μg and 0.1 μg antibody) compared to control vaccinated mice. This demonstrates that partial depletion of Treg at the local site of immunization is sufficient to enhance immune responses to the vaccine antigen. It also suggests that anti-CD25 may function additionally or in some case alternately by mechanisms additional to Treg depletion such as depletion of IL-2 or molecules that bind to IL-2.

### Table 1

Mice were immunized and challenged with 1000 P. berghei (ANKA strain clone 234) sporozoites as previously described. In brief, mice were challenged by i.v. injection in the tail vein with sporozoites dissected from the salivary glands of infected female Anopheles stephensi mosquitoes and homogenized in RPMI 1640 medium. Mice were challenged at 42 or 52 days after they had received anti-CD25 with or without vaccination. Infection was determined by the presence of ring forms in Giemsa stained blood smears taken 7-14 days after challenge Animals were challenged with 1000 sporozoites to provide a stringent liver-stage challenge. Treg depleted groups showed increased protection compared to those not administered antibody or administered the control antibody GL113, *v ¶; P=0.024 Mantel-Haenszel chi-squared test stratified for no. of days post-anti-CD25.

| | | | | No. of |
|---|---|---|---|---|
| | No. | No. in | % | days pos |
| Immunization | protected | group | protected | CD25 |
| GL113 i.p. FP/MVA | 5 | 10 | 50* | 42 |
| aCD25 i.p. FP/MVA | 8 | 10 | 80^{¶} | 42 |
| aCD25 i.p. | 1 | 10 | 10 | 42 |
| FP/MVA | 3 | 10 | 30* | 52 |
| aCD25 i.p. FP/MVA | 4 | 10 | 40^{¶} | 52 |
| [aCD25 + FP]id/MVA | 6 | 9 | 67^{¶} | 52 |
| aCD25 i.p. | 1 | 10 | 10 | 52 |

### REFERENCES

1. Sakaguchi, S. 2003. Control of immune responses by naturally arising CD4+ regulatory T cells that express toll-like receptors. J Exp Med 197:397.
2. Bluestone, J. A., and A. K. Abbas. 2003. Natural versus adaptive regulatory T cells. Nat Rev Immunol 3:253.
3. Bach, J. F., and J. Francois Bach. 2003. Regulatory T cells under scrutiny. Nat Rev Immunol 3:189.
4. Mittrucker, H. W., and S. H. Kaufmann. 2004. Mini-review: regulatory T cells and infection: suppression revisited. Eur J Immunol 34:306.
5. Nagler-Anderson, C., A. K. Bhan, D. K. Podolsky, and C. Terhorst. 2004. Control freaks: immune regulatory cells. Nat Immunol 5:119.
6. Golgher, D., E. Jones, F. Powrie, T. Elliott, and A. Gallimore. 2002. Depletion of CD25+ regulatory cells uncovers immune responses to shared murine tumor rejection antigens. Eur J Immunol 32:3267.
7. Jones, E., M. Dahm-Vicker, A. K. Simon, A. Green, F. Powrie, V. Cerundolo, and A. Gallimore. 2002. Depletion of CD25+ regulatory cells results in suppression of melanoma growth and induction of autoreactivity in mice. Cancer Immun 2:1.
8. Yang, Y., C. T. Huang, X. Huang, and D. M. Pardoll. 2004. Persistent Toll-like receptor signals are required for reversal of regulatory T cell-mediated CD8 tolerance. Nat Immunol.
9. Casares, N., L. Arribillaga, P. Sarobe, J. Dotor, A. Lopez-Diaz de Cerio, I. Melero, J. Prieto, F. Borras-Cuesta, and J. J. Lasarte. 2003. CD4+/CD25+ regulatory cells inhibit activation of tumor-primed CD4+ T cells with IFN-gamma-dependent antiangiogenic activity, as well as long-lasting tumor immunity elicited by peptide vaccination. J Immunol 171:5931.
10. Wood, K. J., and S. Sakaguchi. 2003. Regulatory T cells in transplantation tolerance. Nat Rev Immmol 3:199.
11. Schneider, J., S. C. Gilbert, T. J. Blanchard, T. Hanke, K. J. Robson, C. M. Hannan, M. Becker, R. Sinden, G. L. Smith, and A. V. Hill. 1998. Enhanced immunogenicity for CD8+ T cell induction and complete protective efficacy of malaria DNA vaccination by boosting with modified vaccinia virus Ankara. Nat Med 4:397.
12. Moorthy, V. S., M. Pinder, W. H. Reece, K. Watkins, S. Atabani, C. Hannan, K. Bojang, K. P. McAdam, J. Schneider, S. Gilbert, and A. V. Hill. 2003. Safety and immunogenicity of DNA/modified vaccinia virus ankara malaria vaccination in African adults. J Infect Dis 188:1239.
13. McConkey, S. J., W. H. Reece, V. S. Moorthy, D. Webster, S. Dunachie, G. Butcher, J. M. Vuola, T. J. Blanchard, P. Gothard, K. Watkins, C. M. Hannan, S. Everaere, K. Brown, K. E. Kester, J. Cummings, J. Williams, D. G. Heppner, A. Pathan, K. Flanagan, N. Arulanantham, M. T. Roberts, M. Roy, G. L. Smith, J. Schneider, T. Peto, R. E. Sinden, S. C. Gilbert, and A. V. Hill. 2003. Enhanced T-cell immunogenicity of plasmid DNA vaccines boosted by recombinant modified vaccinia virus Ankara in humans. Nat Med 9:729.
14. Anderson, R. J., C. Hannan, S. Gilbert, S. M. Laidlaw, E. Sheu, S. Korten, R. Sinden, G. Butcher, M. A. Skinner, and A. Hill. 2003. Enhanced CD8+ T-cell immune responses and protection elicited against Plasmodiun berghei malaria by prime boost immunisation regimes using a novel attenuated fowlpox virus. J Immunol: Accepted.
15. Plebanski, M., and A. V. Hill. 2000. The immunology of malaria infection. Curr Opin Immunol 12:437.
16. Kursar, M., K. Bonhagen, J. Fensterle, A. Kohler, R. Hurwitz, T. Kainmradt, S. H. Kaufmarn, and H. W. Mittrucker. 2002. Regulatory CD4+CD25+T cells restrict memory CD8+ T cell responses. J Exp Med 196:1585.
17. Phan, G. Q., J. C. Yang, R. M. Sherry, P. Hwu, S. L. Topalian, D. J. Schwartzentruber, N. P. Restifo, L. R. Haworth, C. A. Seipp, L. J. Freezer, K. E. Morton, S. A. Mavroukakis, P. H. Duray, S. M. Steinberg, J. P. Allison, T. A. Davis, and S. A. Rosenberg. 2003. Cancer regression and autoimmunity induced by cytotoxic T lymphocyte-associated antigen 4 blockade in patients with metastatic melanoma. Proc Natl Acad Sci USA 100:8372.
18 Suvas S, Kumaraguru U, Pack C D, Lee S, Rouse B T. 2003. CD4+CD25+T cells regulate virus-specific primary and memory CD8+ T cell responses. J Exp Med 198(6):889-901.
19 Aandahl E M, Michaelsson J, Moretto W J, Hecht F M, Nixon D F. 2004. Human CD4+CD25+ regulatory T cells control T-cell responses to human immunodeficiency virus and cytomegalovirus antigens. J Virol. 78(5):2454-9.

1. A method of inducing an immune response in an organism, comprising administering a vaccine to the organism under conditions in which the function of Treg cells is impaired.

2. The method according to claim 1, wherein the Treg cells in the organism have been depleted.

3. The method according to claim 1, wherein the conditions in which the function of Treg cells is impaired using an agent that impairs the function of Treg cells.

4. The method according to claim 3, wherein said agent reduces the activity of CD25.

5. The method according to claim 4, wherein said agent disrupts the interaction between CD25 (IL-2R alpha) and one or more of its ligands.

6. The method according to claim 4, wherein said agent is soluble CD25 or anti-CD25 antibody.

7. The method according to claim 3, wherein said agent reduces the activity of one or more of CTLA-4, GITR, IL-10, TGF-β or FoxP3.

8. A method for impairing Treg cell function in an organism, comprising administering to the organism an agent that impairs the function of Treg cells.

9. The method according to claim 3, wherein said agent depletes Treg cells.

10. The method according to claim 1 where the vaccine is viral vectored vaccine, a subunit vaccine, or an attenuated microbial vaccine.

11. A method of inducing an immune response in an organism, comprising administering to the organism to a priming composition that comprises a vectored vaccine comprising an antigen, and administering to the organism a boosting composition comprising a vectored vaccine comprising the same antigen in the priming composition, wherein the function of Treg cells in said organism is impaired prior to or at substantially the same time the priming composition is administered.

12. (canceled)

13. (canceled)

14. A method of enhancing an immune response in an organism, comprising administering a vaccine to the organism in the presence of an anti-CD25 antibody.

15. A method of enhancing an immune response in an organism, comprising administering a vaccine to the organism in the presence of an agent that disrupts the interaction between CD25 (IL-2R alpha) and one or more of its ligands.

16. The method according to claim 15, wherein said agent is soluble CD25 or anti-CD25 antibody.

17. The method according to claim 15, wherein the immune response is an antigen-specific immune response.

18. The method according to claim 17, wherein the antigen-specific immune response is a CD8+ T cell response, CD4+ T cell response, or antibody response.

19. (canceled)

20. The method according to claim 15, wherein the vaccine is a vectored vaccine.

21. The method according to claim 15, wherein the vaccine is a subunit vaccine.

22. The method according to claim 15, wherein the vaccine comprises non-self antigen.

23. The method according to claim 15, wherein the vaccine comprises a recombinant antigen.

24. The method according to claim 20, wherein the vectored vaccine is a viral vectored vaccine.

25. The method according to claim 20, wherein the vectored vaccine is a non-viral vector or a non-replicating or replication-impaired viral vector.

26. The method according to claim 1, wherein the viral vectored vaccine is a vaccinia virus vector, avipox vector, or canarypok vector, or a non-replicating adenovirus.

27. The method according to claim 9, wherein the agent for impairing Treg cell function by depleting Treg cells is an anti-CD25 antibody, antigen-binding fragment of an anti-CD25 antibody, or a fusion protein comprising IL-2 fused to a toxin.

28. The method according to claim 9, wherein the agent for depleting Treg cells is an anti-CD25 antibody.

29. The method according to claim 9, wherein the vaccine is administered substantially simultaneously with, or after the administration of the agent that depletes Treg cells.

30. The method according to claim 9, wherein the vaccine is co-administered with the agent that depletes Treg cells.

31. The method according to claim 29, wherein the vaccine is administered at least one day after administration of the agent that depletes Treg cells.

32. The method according to claim 29, wherein the vaccine is administered less than about 70 hours after administration of the agent that depletes Treg cells.

33. The method according to claim 1, wherein the organism is a vertebrate.

34. The method according to claim 1, wherein inducing the response is therapeutic or prophylactic.

35. The method according to claim 1, where the vaccine is administered by injection.

36. A kit, comprising:
i. an agent that impairs Treg cell function when administered to an organism;
ii. a priming composition comprising one or more antigens encoded by a non-replicating or replication-impaired recombinant viral vector; and,
iii. a boosting composition comprising one or more antigens encoded by a non-replicating or replication-impaired recombinant viral vector, wherein at least one antigen is the same antigen of the priming composition.

37. A method for generating an immune response against at least one antigen, comprising administering at least one dose of an agent that impairs Treg cell function substantially simultaneous with or followed by at least one dose of a priming composition comprising one or more antigens encoded by a non-replicating or replication-impaired recombinant viral vector, followed by at least one dose of a boosting composition comprising or more antigens encoded by a non-replicating or replication-impaired recombinant viral vector, wherein at least one antigen which is the same antigen of the priming composition.

38. (canceled)

39. (canceled)

40. The method of claim 26, wherein the vaccinia virus vector is MVA or NYVAC.

41. The method of claim 26, wherein the avipox vector is fowlpox.

42. The method of claim 27, wherein the toxin is diphtheria toxin.

43. The method of claim 27, wherein the anti-CD25 antibody, antigen-binding CD25 antibody fragment, or a fusion protein comprising IL-2 fused to a toxin are expressed by a vector.
